(19) United States
(12) Patent Application Publication
(10) Pub. No.: US 2007/0016140 A1
(43) Pub. Date: Jan. 18, 2007

(54) Exchangeable safety needle assembly

(75) Inventor: Barry B. Berler, Huntingdon Valley, PA (US)

Correspondence Address: MILLER LAW GROUP, PLLC, 25 STEVENS AVENUE, WEST LAWN, PA, 19609, US

(73) Assignee: Devon Safety Products, Inc.

(21) Appl. No.: 11/443,996

(22) Filed: May 31, 2006

Related U.S. Application Data
(60) Provisional application No. 60/688,938, filed on Jun. 9, 2005.
(60) Provisional application No. 60/717,085, filed on Sep. 14, 2005.

(51) Int. Cl. A61M 5/32

(52) U.S. Cl. 604/198

(57) ABSTRACT

A safety needle assembly has a rigid outer housing that encloses a locking mechanism cooperable with an apparatus that slides a sheath over the tip of the needle to prevent accidental sticks after the needle has been used. The safety needle assembly presents a common housing diameter that can be connected to syringe barrels formed with a suitably sized connection port irrespective of the size of the syringe barrel. The safety needle assembly is interchangeable with respect to properly configured syringe barrels. The plunger in the syringe barrel is configured to actuate the locking mechanism within the safety needle assembly when the plunger reaches the end of the stroke to the bottom of the barrel. The outer housing is formed with internal threads that engage corresponding external threads on the connection port to detachably mount the safety needle assembly on the syringe barrel.

### CROSS-REFERENCE TO RELATED APPLICATIONS

This application claims domestic priority on U.S. Provisional Patent Application Ser. No. 60/688,938, filed Jun. 9, 2005, and on U.S. Provisional Patent Application Ser. No. 60/717,085, filed Sep. 14, 2005, the contents of both provisional applications being incorporated herein by reference.

### FIELD OF THE INVENTION

The present invention relates generally to safety syringes of the type to cover the point of the needle after use to prevent accidental sticks with a contaminated needle, and, more particularly, to a safety needle that can be used on different syringe barrels.

### BACKGROUND OF THE INVENTION

Safety syringes are known in the art, such as depicted in U.S. Pat. No. 5,720,727, issued to Gary Alexander on Feb. 24, 1998, the sheath is advanced by operation of the plunger. While such a configuration provides an excellent protection for health care workers from accidental needle sticks, the mechanism does not prevent intravenous drug users from overriding the safety features and re-using the needle for illicit drug purposes, exposing the user to risks associated with contaminated needles. Most known locking mechanisms are designed to secure the syringe in an exposed condition. Such locking mechanisms are not designed to prevent a determined drug user from overriding the locking mechanism to permit the plunger to be retracted from the barrel in order to re-use the needle.

An improvement in the locking mechanism for a safety needle can be found in Applicant's U.S. Pat. No. 6,626,863, granted Sep. 30, 2003. In this configuration, the needle is housed within a tubular sheath that is movable between an exposed position, in which the point of the needle is extended beyond the sheath for utilization, and a covered position, in which the point is withdrawn into the sheath. The movement of the sheath is actuated by engagement with the syringe plunger that moves a locking mechanism into a position that slides the sheath over the needle. The barrel of the syringe can also incorporate an additional locking mechanism that prevents the plunger from being retracted out of the barrel, and thus perhaps exposing the needle from the sheath.

Safety syringes incorporating the aspects of the '863 patent have been available commercially and are provided as an integral unit, including the barrel, plunger, needle, sheath and locking mechanism. Once used, the safety syringe cannot be re-used. Furthermore, since the needle cannot be removed from the syringe barrel, the size or gauge of needle is not variable with respect to the size of the barrel being selected for use, except that different combinations of barrel volume and needle gauge can be provided for use by the appropriate health care worker.

In the manufacture of safety syringes, a number of different needle gauges must be provided. Conventional manufacturing techniques provide a different barrel size in each available needle gauge. For a safety syringe of the type shown and described in the aforementioned U.S. Pat. No. 6,626,863, each gauge of needle requires a different housing bore and a suitably sized diaphragm through which the needle passes to be in flow communication with the medicine being dispensed from the barrel of the syringe. Such multiple needle sizes for each barrel size requires many different parts and greatly increases the manufacturing complexity in providing safety needles for use by the health care industry.

Syringes and disposable needles are manufactured in many different sizes, i.e. diameters and/or capacity of the syringe barrel, and in several different needle gauges for each different barrel diameter. To provide a standardized diameter for an attachable needle assembly to be mounted on the distal end of the syringe barrel, the syringe barrel would have to be configured to accept the standardized housing diameter for mounting thereon. In association with the configuration of the syringe barrel, the plunger must be operable to expel all of the fluid material from within the syringe barrel when the plunger is completely depressed. Lastly, in the instances where a safety needle assembly having a locking mechanism is utilized, the plunger must be operable to activate the locking mechanism when the plunger is completely depressed.

The needle locking mechanism depicted in the aforementioned U.S. Pat. No. 6,626,863, is somewhat complex and difficult to manufacture and assemble. An improvement in the apparatus that affects a locking of the sheath in the extended position to cover the sharp tip of the needle is preferred. A reduction in the complexity of the safety needle assembly would result in lower manufacturing costs, and a resultant lower retail cost to the health care industry. By simplifying the needle locking mechanism, the complexity of the safety needle assembly would be significantly reduced.

Since health care practitioners often fill the syringe barrel with the desired medication with a large bore needle to decrease the length of time to fill the syringe barrel, and then substitute a smaller gauge needle for inserting the needle into the patient's body to reduce the pain associated with the stick of the needle, the health care practitioner may mount one needle on the syringe barrel, then remove the first needle by untwisting the mounting from the receptor on the end of the syringe barrel and add a second needle to the barrel receptor. Each manipulation of the needle presents an independent risk for an accidental stick into the health care practitioner. Thus, any improvement in the handling of the needle and/or syringe would be advantageous.

Accordingly, it would be desirable to provide the housing of the safety needle assembly with a convoluted outer surface to improve the ability to grip the housing for either mounting or removing the safety needle assembly on the syringe barrel receptor.

It would also be desirable to provide a common needle assembly that could be utilized with a plurality of syringe barrels that would enable the same needle assembly to be used with any size syringe barrel.

It would also be desirable to provide additional improvements to the safety needle assembly that would reduce the number of individual components needed for the manufacture of a safety needle assembly that could be selectively mounted on a separate syringe barrel.

Accordingly, it would be desirable to provide a needle locking assembly that would be effective in the locking of the sheath in the extended position while reducing the number of parts and the complexity of manufacture of the safety needle assembly.

### SUMMARY OF THE INVENTION

It is an object of this invention to provide a safety needle assembly that can be selectively mounted on a syringe barrel adapted to connect with the safety needle assembly.

It is a feature of this invention that the safety lock mechanism preventing a used needle from being reused is incorporated into the safety needle assembly.

It is an advantage of this invention that the needle assembly incorporates a safety lock preventing the needle assembly from being reused.

It is another object of this invention to form the safety needle assembly with an outer housing that is detachably connectable to a syringe barrel.

It is another feature of this invention that the outer housing of the safety needle assembly is formed with threads to facilitate the connection of the assembly to a correspondingly formed syringe barrel.

It is another advantage of this invention that the Sharps disposal of the needle assembly can be reduced in volume by disconnecting the used needle assembly from the syringe barrel.

It is still another feature of this invention that the needle is associated with a slidable sheath that becomes positioned over the point of the needle when the syringe plunger reaches the bottom of the barrel.

It is still another advantage of this invention that the sheath-covered needle no longer presents a hazard to the health care worker as the point of the needle is covered and occluded by the sheath.

It is yet another feature of this invention that the safety needle assembly includes a movable locking mechanism that is engaged by the syringe plunger to activate the sliding movement of the sheath over the needle.

It is yet another advantage of this invention that the number of individual components for the construction of safety needles is reduced to greatly reduce the complexity in the manufacturing of the safety needle assemblies.

It is still another object of this invention to provide a safety needle assembly that can be mounted on different sized syringe barrels.

It is a further advantage of this invention that the safety needle assembly can be a common apparatus that is applicable to different sized syringe barrels to enhance the flexibility of the use of the safety needle assemblies.

It is a further feature of this invention that the syringe barrels are formed with an attachment port for connection to the safety needle assembly.

It is still a further feature of this invention that the attachment port has the same size irrespective of the size of the syringe barrel.

It is yet a further feature of this invention that the plunger is adapted to actuate the locking mechanism within the attached safety needle assembly when the plunger reaches the end of the stroke within the syringe barrel.

It is still a further advantage of this invention that the locking mechanism is not activated unless the plunger is fully depressed into the syringe barrel, corresponding to the expelling of all the medicine dispensed into the syringe barrel.

It is still another object of this invention to provide a safety needle assembly for selective use on different sized syringe barrels which is durable in construction, inexpensive of manufacture, facile in assemblage, and simple and effective in use.

These and other objects, features and advantages are accomplished according to the instant invention by providing a safety needle assembly having a rigid outer housing that encloses a locking mechanism cooperable with an apparatus that slides a sheath over the tip of the needle to prevent accidental sticks after the needle has been used. The safety needle assembly presents a common housing diameter that can be connected to syringe barrels formed with a suitably sized connection port irrespective of the size of the syringe barrel. The safety needle assembly is interchangeable with respect to properly configured syringe barrels. The plunger in the syringe barrel is configured to actuate the locking mechanism within the safety needle assembly when the plunger reaches the end of the stroke to the bottom of the barrel. The outer housing is formed with internal threads that engage corresponding external threads on the connection port to detachably mount the safety needle assembly on the syringe barrel.

### BRIEF DESCRIPTION OF THE DRAWINGS

The foregoing and other objects, features, and advantages of the invention will appear more fully hereinafter from a consideration of the detailed description that follows, in conjunction with the accompanying sheets of drawings. It is to be expressly understood, however, that the drawings are for illustrative purposes and are not to be construed as defining the limits of the invention.

FIG. 1 is an elevational view of the safety needle assembly incorporating the principles of the instant invention, the outer housing being broken away to show the actuator and lock mechanism within the safety needle assembly;

FIG. 2 is a cross-sectional view of the safety needle assembly taken along the longitudinal centerline to reveal the internal configuration of the actuator and lock mechanism depicted in FIG. 1;

FIG. 3 is a cross-sectional view of an assembled syringe having a safety needle assembly mounted thereto according to the principles of the instant invention, the plunger being depicted near the end of the stroke to the bottom of the syringe barrel;

FIG. 4 is a cross-sectional view of an assembled syringe having a safety needle assembly mounted thereto, similar to that shown in FIG. 3, but with the actuator engaged to move the sheath over the tip of the needle to prevent accidental sticks;

FIG. 5 is an elevational view of the sheath head mounted within the outer housing member of the safety needle assembly;

FIG. 6 is an elevational view of the sheath head oriented orthogonally to the elevational view of FIG. 5;

FIG. 7 is a cross-sectional view of the sheath head taken along the longitudinal centerline of the sheath head looking in the same orientation as FIG. 6;

FIG. 8 is an end view of the sheath head corresponding to a projection of FIG. 7;

FIG. 9 is an elevational detail view of the outer housing of the safety needle assembly;

FIG. 10 is a cross-sectional view of the housing shown in FIG. 9;

FIG. 11 is a cross-sectional view of the housing looking orthogonally to the view of FIG. 10;

FIG. 12 is an end view of the outer housing oriented as a projection of the outer casing as oriented in FIG. 11;

FIG. 13 is an elevational view of the diaphragm mounted on the end of the sheath head within the safety needle housing;

FIG. 14 is a elevational schematic view of the assembled components of the safety needle assembly looking orthogonally to the views of FIGS. 1 and 2, the needle being removed for purpose of clarity, the actuator being shown in phantom lines, the central needle support being depicted in dashed lines and the diaphragm being shown in broken lines within the outer housing;

FIG. 15 is an elevational view of a 3 ml syringe barrel adapted for mounting the safety needle assembly as depicted in FIGS. 1 and 2, a plunger incorporating the principles of the instant invention being shown disposed within the syringe barrel;

FIG. 16 is an elevational view of a 5 ml syringe barrel having a connection port at the end of the barrel for mounting the safety needle assembly;

FIG. 17 is an elevational view of a 10 ml syringe barrel having a connection port for the mounting of the safety needle assembly; and

FIG. 18 is an elevational view of a 30 ml syringe barrel having a connection port for the mounting of the safety needle assembly, a plunger incorporating the principles of the instant invention being shown disposed within the syringe barrel.

### DETAILED DESCRIPTION OF THE PREFERRED EMBODIMENT

Referring to the drawings, the components of a safety needle assembly incorporating the principles of the instant invention can best be seen. The safety needle assembly 10 includes an outer housing 12 including a collar 13 threaded for quick connection to the receptor on a suitably configured syringe barrel. The diaphragm 15 is mounted within the housing 12 to provide a fluid-tight seal against the housing 12 and has an actuator side 16, facing the syringe barrel 50, and a needle side 17 from which the needle 11 projects. The needle 11 is received through the diaphragm 15, passing through a bore 44 in a central needle support 40 and projecting outwardly therefrom in an axial manner with respect to the housing 12.

The safety needle assembly 10 further includes an actuator 20 which includes a sheath head 21 through which the needle 11 passes and is extendible therefrom. The sheath head 21 includes a body portion 22 and a pair of axially extending legs 24 that project toward the syringe barrel 50 from the body portion 22 and engage with the diaphragm 15, as will be described in greater detail below. The sheath 25 through which the needle 11 passes is attached to the body portion 22 to form an integrally movable assembly 20 that allows the sheath 25 to extend over the needle 11 to cover the tip 11a of the needle 11 to prevent exposure thereof after the plunger 55 in the syringe barrel 50 has completely expelled the medicine therein through the needle 11 into the patient.

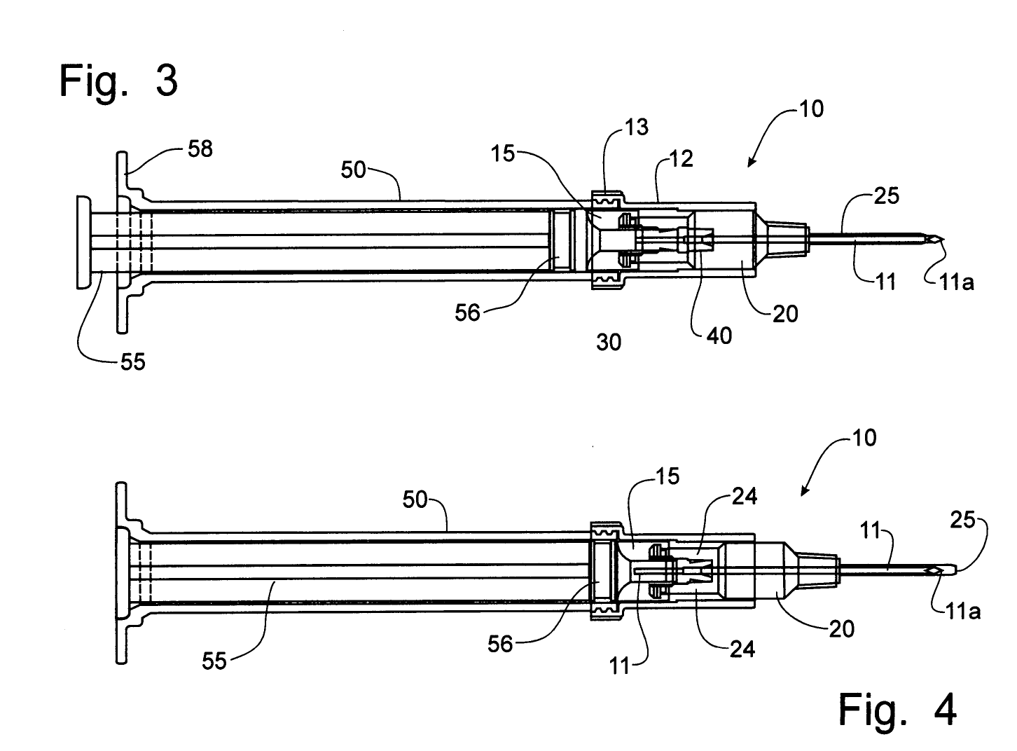

The actuator 20 is movable between an exposed position, as seen in FIG. 3, in which the needle tip 11a projects outwardly from the sheath member 25 and is in an operable position for being inserted into an object, such as an IV line or a human patient. Once actuated, the sheath member 25 is movable into a covered position, best seen in FIG. 4, in which the needle 11 is completely covered by the sheath member 25 such that the tip 11a of the needle 11 is prevented from accidentally injuring someone.

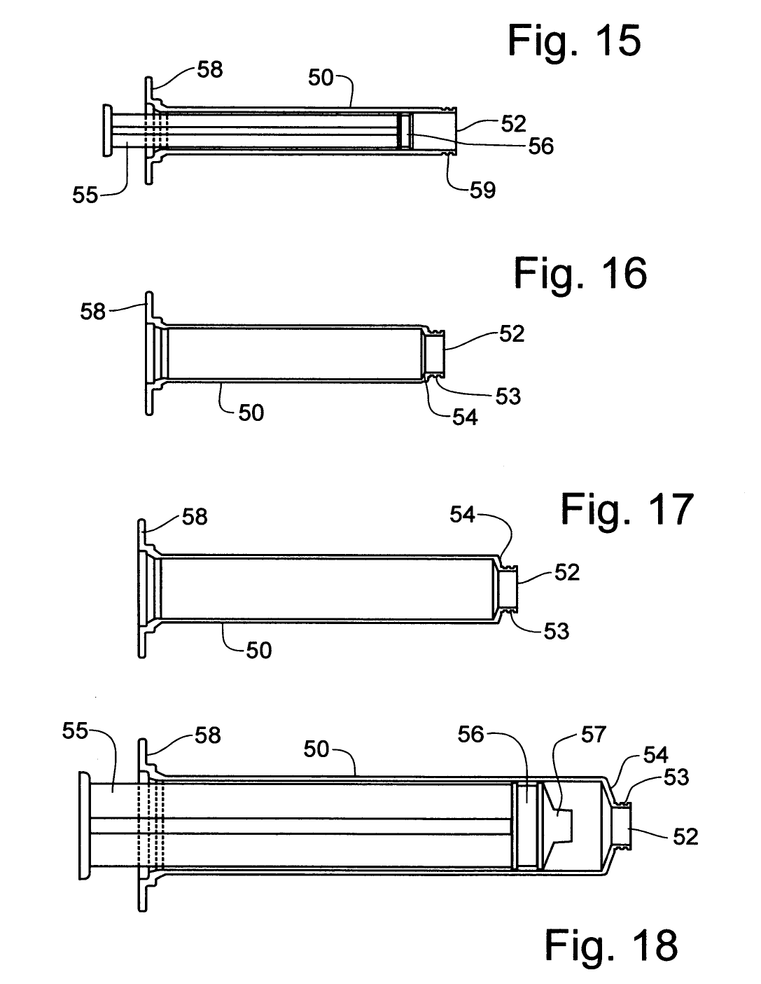

The collar 13 is formed with threads 14, which are preferably located on the interior side of the collar 13 to be engagable with corresponding threads on a syringe barrel 50 located on the exterior side thereof to permit the safety needle assembly 20 to be quickly coupled to the syringe barrel 50 to form an operable safety syringe. The collar 13 is sized to mate directly on a 3 ml syringe barrel, as is depicted in FIG. 15. Syringe barrels 50 having a larger diameter than the standard 3 ml syringe barrel 50 are formed with a connection port 52 having the same external diameter as the end of the 3 ml syringe barrel 50. A sloped transition portion 54 reduces the diameter of the end of the syringe barrel 50 to the 3 ml size of the connection port 52. The connection between the collar 13 and the connector port 52 on the syringe barrel 50 can be in the form of a Luer Lock or any other appropriate quick connect apparatus that is known in the art.

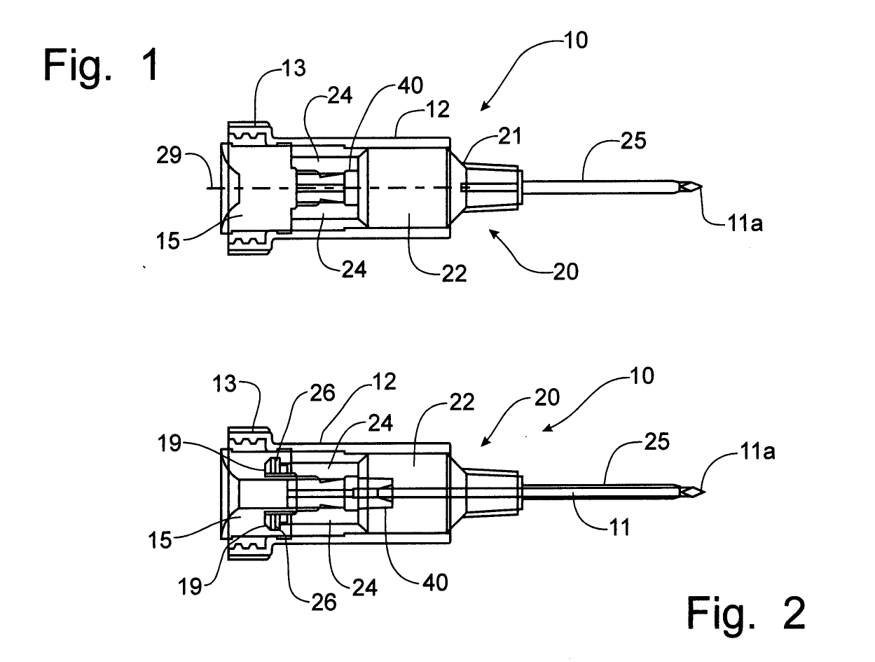

The safety needle assembly 20 incorporates a locking mechanism 30 that fixes the actuator 20 in the covered position. Preferably, the locking mechanism 30 is formed from a cam member 31 formed on the interior side of each of the actuator legs 24. The cam member 31 includes a ramp portion 32 angled inwardly toward the syringe barrel 50, as can be seen best in FIGS. 1, 2, 7 and 8, that terminates in a lock surface 33 oriented generally perpendicularly to the longitudinal centerline 19 of the safety needle assembly 10. The cam members 31 are engagable with the central needle support 40, as will be described in greater detail below, such that the ramp portions 32 deflect the cam member 31 outwardly until the lock surface 32 passes by the central needle support 40, whereupon the cam members 31 retract inwardly positioning the lock surfaces 33 under the central needle support 40. This positioning of the lock surfaces 33 under the central needle support 40 occurs as the plunger 55 forces the diaphragm 15 into the housing 12, as will be described in greater detail below.

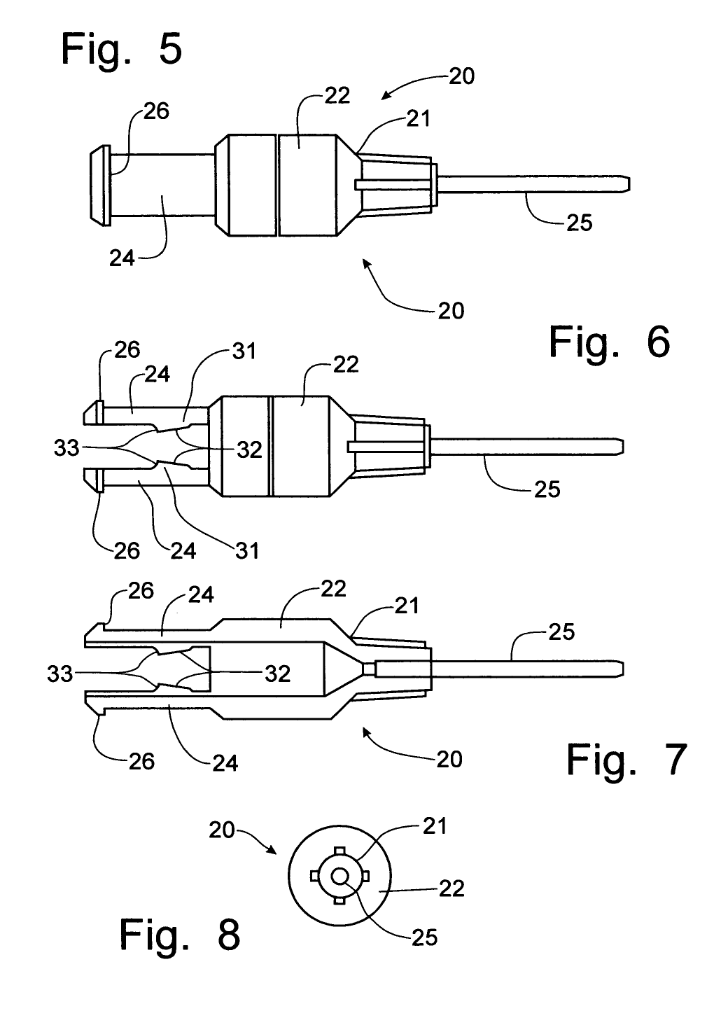

As is best seen in FIG. 5, the collar 13 is preferably formed with longitudinally extending ribs 39 to facilitate the gripping of the housing 12 of the safety needle assembly 10. Since the housing 12 must be rotated to thread the safety needle assembly 10 onto the barrel connector 52, the ribs 39 provide a positive gripping surface for engagement by the health care practitioner's fingers. The ribs 39 are particularly advantageous when trying to remove the safety needle assembly from the syringe barrel as the housing may be tightened to the point of being difficult to grasp. Furthermore, if the health care practitioner's fingers are wet, the gripping of a smooth housing surface is unlikely to provide sufficient torque on the housing 12 to affect whether tightening or loosening.

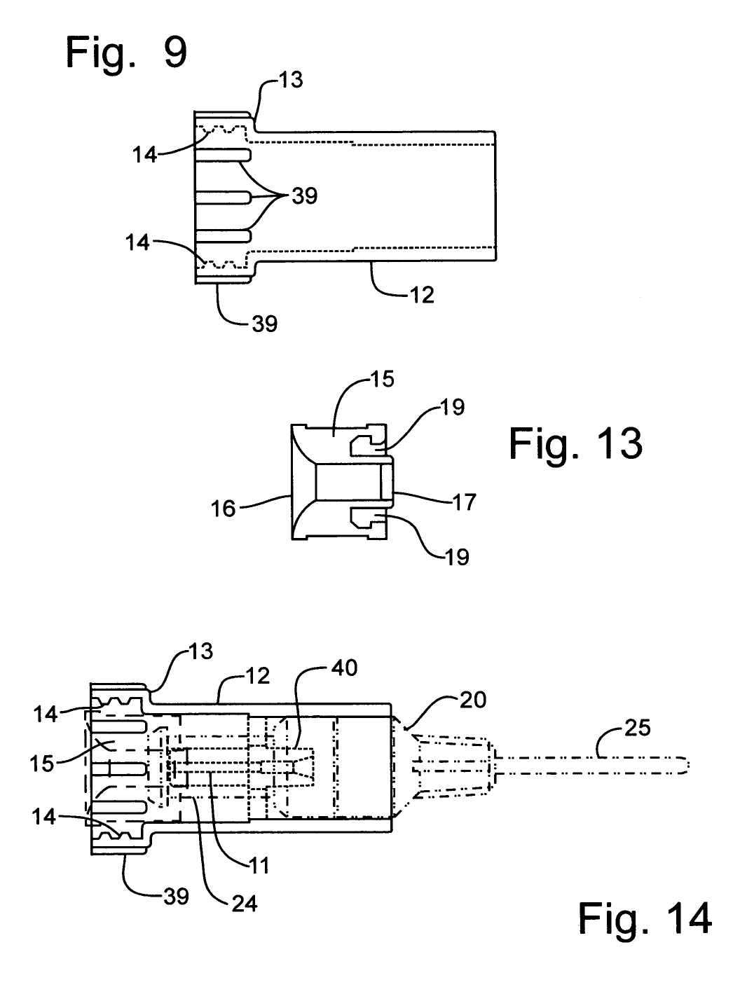

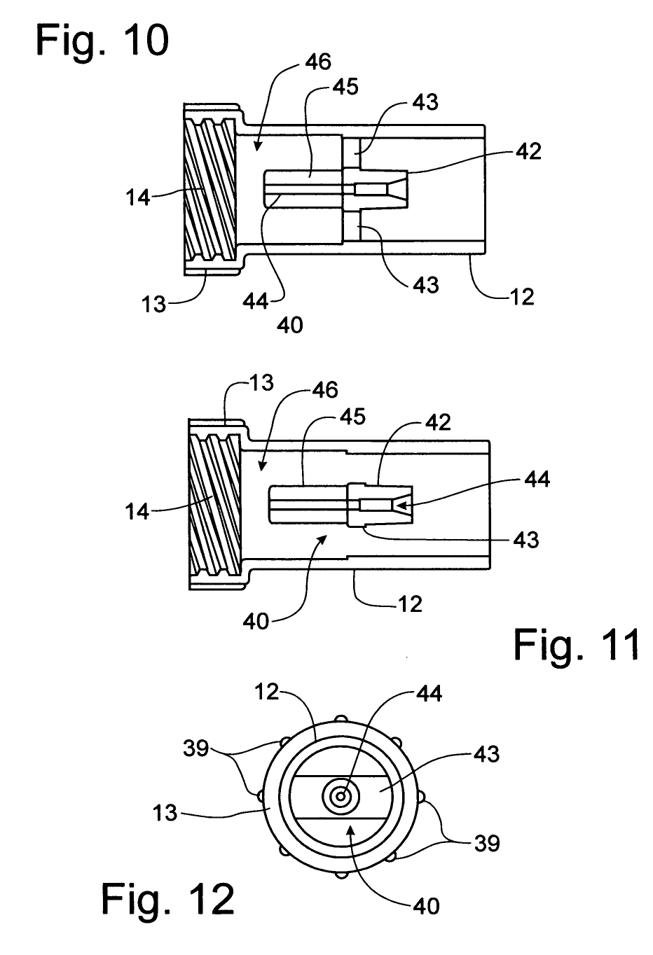

Referring to the housing 12 shown in FIGS. 9-12, the housing 12 is formed with a central needle support 40 along the longitudinal centerline 29 of the housing 12. The central needle support 40 receives the needle through a bore 44 passing along the longitudinal centerline 29 through the central needle support 40 for passage into the diaphragm 15. The central needle support 40 comprises a lower portion 42 supported on a bridge 43 extending across the diameter of the housing 12 and an upper portion 45. The upper portion 45 projects above the bridge 43 as an extension of the lower portion 42. The bore 44 extends continuously through the upper and lower portions 42, 45 of the central needle support 40.

The upper portion 45 of the central needle support 40 defines a tubular mount at the center of an upper chamber 46 of the housing 12. The diaphragm 15 is mounted for axial movement along the housing on the upper portion 45 which forms a central mount for the diaphragm 15. As noted above, the needle 11 passes through the bore 44 into the diaphragm 15 to be in flow communication with the syringe barrel 50. By standardizing the size of the housing 12, and by standardizing the outer diameter of the upper portion 45 forming the central mount in the upper chamber 46, the size and shape of the diaphragm 15 can also be standardized to reduce substantially the number of different parts required to make safety needle assemblies 10 with different needle gauges.

The bore 44 will be sized to match the gauge of the needle being utilized in the safety needle assembly, but the magnitude of the diameter of the bore 44 has no bearing on the outer diameter of the upper portion 45 and, thus, the size and shape of the diaphragm 15 can be standardized to fit within the upper chamber 46. While the outer dimensions of the housing 12 will not vary, the diameter of the bore 44 will vary according to the gauge of the needle 11 being utilized in the safety needle assembly 10. Nevertheless, the formation of the housing 12 can be common with all needle gauges until the bore 44 is drilled into the central needle support 40. Furthermore, the utilization of the upper portion 45 of the central needle support 40 provides additional stability for the needle 11, as the longer support member 40 through which the bore 44 extends reduces the ability of the needle 11 to move laterally within the safety needle assembly 10.

Further commonizing the syringe barrel 50 is necessary for the utilization of a common diameter safety needle assembly. As noted above, the outer housing 12 is sized to fit on the smallest commonly used syringe in medical practice, the 3 ml syringe. Accordingly, as can be seen in FIG. 15, the 3 ml syringe barrel 50 is formed simply with a grip portion 58 at the upper end thereof and a threaded outer surface 59 at the lower end of the syringe barrel 50. Otherwise, the barrel 50 is open at the lower end. Typically, the syringe barrel 50 will be shipped in a sterilized package with a suitable sized plunger 55 inserted into the barrel 50. The syringe barrel 50 could be formed with a barrel lock device (not shown) that fixes the plunger 55 in the barrel 50 when the plunger 55 reaches the end of the stroke at the bottom of the barrel 50. One skilled in the art will recognize that the use of a barrel lock (not shown) is not needed for proper operation of the safety needle assembly 10 as will be described in greater detail below.

As reflected in FIGS. 16-18, syringe barrels 50 that have a larger capacity than 3 ml can also be manufactured to accept the mounting of a safety needle assembly 10. More particularly, the open lower end of the syringe barrel 50 can be formed with a connection port 52 that has the same diameter as the 3 ml barrel 50. To carry a greater supply of medicine, the general diameter of the barrel 50 is larger than the 3 ml barrel. However, the lower end of the barrel 50 can be formed with a connection port 52, as is depicted in FIGS. 16-18, that will accept the standardized size of the housing 12. A sloped transition portion 54 reduces the diameter of the barrel 50 from the sized necessary to define the appropriate capacity of the barrel to the common 3 ml size of the connection port 52. The transition portion 54 is sloped, instead of perpendicular to the centerline 29 of the barrel 50, so that the medicine within the barrel 50 can be completely dispensed to the patient when the plunger 55 is completely depressed into the syringe barrel 50.

Each plunger 55 can be formed to tailor-fit each syringe barrel 50 in terms of diameter of the barrel 50 and in terms of mating with the transition portion 54 so that all of the medicine in the syringe barrel 12 will be dispensed when the plunger 55 is completely depressed. As can be seen in FIGS. 15 and 18, the plunger 55 is shaped to mate with the transition portion 54 of the barrel 50, and includes an actuation tip 57 that extends into the housing 12 of the safety needle assembly 10 to move the diaphragm 15 as will be described in greater detail below.

Assembly of the safety needle assembly requires the formation of a standard housing 12, a standard diaphragm 15, a needle 11 having a selected gauge, and an actuator 20, including the body portion 22 with the legs 24 extending axially therefrom and a sheath member 25 affixed to the body portion 22 and extending in the opposite direction from the legs 24. The actuator 20 is mounted into the housing 12 with the legs 24 extending on opposing sides of the bridge 43. The tips of the legs 43 are formed in a shape, as is best seen in FIGS. 5-7, having a protruding lip 36 that is sized to mate with a matched cavity 19 formed in the diaphragm 15. Thus, the legs 24 are engaged into the cavities 19 in the diaphragm 15 so that the diaphragm 15 is mounted on the actuator 20. The diaphragm 15 is located in the upper chamber 46 of the housing 12 and mounted on the central mount 45 to be movable axially along the central mount 45. The needle 11 is passed through the sheath member 25 and the body portion 22 of the sheath head 21, passing through the appropriately sized bore 44 and through the diaphragm 15 to be in flow communication with the barrel 50 when mounted the safety needle assembly 10 is mounted thereon.

In operation, the health care worker would select the barrel 50 having a plunger 55 with a rubber seal 56 positioned within the barrel 50, with the barrel 50 being open at the distal end where the connector 52 is located with threads 53 thereon being located preferably on the exterior surface thereof. The safety needle assembly 10 is then selected with the needle 11 protected by a safety cap (not shown). The collar 13 having the threads 14 (or outwardly projecting ribs as in a Luer Lock) positioned preferably on the interior surface thereof is then engaged with the connector 52 on the barrel 50 and twisted into a locking engagement therebetween. Once the safety cap is removed, the assembly safety syringe with detachable safety needle assembly 10 is ready for utilization.

As with conventional syringes, the assembled two-piece safety syringe is operated by withdrawing the plunger 55 to affect the intake of liquid medicine into the barrel 50. Once air has been ejected from the barrel 50, the needle 11 and tubular sheath member 25 are inserted into the selected target. Depressing the plunger 55 expels the liquid medicine through the needle 11 and into the selected target until the plunger 55 bottoms out against the diaphragm 15. At this point, the plunger 55 is not completely inserted into the barrel 50, as some additional linear movement of the plunger 55 remains available to actuate the locking mechanism 30.

This last movement of the plunger 55 drives the diaphragm 15 axially along the central mount 45 deeper into the upper chamber 46 of the housing 12. The axial movement of the diaphragm 15, due to the engagement of the legs 24 in the cavities 19 of the diaphragm 15, pushes the actuator 20 along the longitudinal center axis 29 of the assembly 10 outwardly to move the tubular sheath member 25 into the covered position over the needle tip 11a. The locking mechanism 30 engages when the actuator 20 has moved into the covered position, which preferably corresponds to the plunger 55 bottoming out into the barrel 50.

The locking mechanism 30 is engaged due to the axial movement of the actuator 20 in response to the plunger 55 driving the diaphragm 15 deeper into the upper chamber 46 of the housing 12. The cam members 31 formed on the interior side of the legs 24 move axially with the rest of the actuator 20, with the sloped ramp portions 32 springing the legs 24 slightly outwardly as the cam members 31 pass the bridge 43 supporting the central needle support 40 within the housing 12. Once the lock surfaces 33 pass below the bridge 43, the legs 24 spring back into position driving the lock surfaces 33 under the bridge 43, which will restrict the return of the cam members 31, and then the actuator 20 back past the bridge 43.

With the needle 11 covered, the needle 11 and tubular sheath member 25 are withdrawn from the selected target. Since the locking mechanism 30 prevents the actuator 20 from moving out of the covered position, the point 11a of the needle 11 will not be exposed to present an opportunity for an accidental stick into the health care practitioner, or to present an opportunity for the safety needle assembly 10 to be re-used, whether remaining engaged with the barrel 50, or removed from the barrel 50.

The safety needle assembly 10 is intended for use on a syringe barrel 50 configured as shown in FIGS. 15-18 with a connector port 52 having threads 53 preferably formed on the exterior surface thereof to mate with the threads 14 on the interior surface of the collar 13 of the safety needle assembly 10. The size or gauge of the safety needle assembly 10 is preferably maintained at a given configuration, such as is conventional for a 3 cc syringe, so that production of the safety needle assembly 10 can be economical. Rather than form the safety needle assembly 10 with different sized housings 12 and collars 13 for connection to the correspondingly different sized syringe barrels 50, having conventional capacities of between 3 cc and 60 cc, the housing 12 and collar 13 are formed at a standard size and the barrels 40 of the syringes are formed to mate with the standardized safety needle assembly 10.

As can be seen in FIGS. 16-18, the barrels 40 having a capacity greater than the conventional 3 cc syringe size are formed with a transition portion 54 that reduces the diameter of the barrel 50 from the conventional diameter corresponding to the particular sized syringe being utilized to the connector port 52 diameter corresponding to the standardized safety needle assembly 10. This transition portion 48 is formed with angularly sloped walls that span between the larger diameter of the large syringe barrel 50 to the standardized connector 52 diameter.

The plunger 55 must then be formed with a mating configuration that closes against the sloped sides of the transition portion 54 when the plunger 55 is completely depressed into the barrel 40 so that all of the fluid material within the barrel 40 is expelled from the barrel 50 through the needle 11. The seal 56 at the end of the plunger 55 will be formed with a tapered tip 57 that will extend into the housing 12 of the attached safety needle assembly 10 to actuate the locking mechanism 30 therein by engaging the diaphragm 15 when the plunger 55 has been fully depressed into the barrel 50. The tapered shape of the tip 57 allows the last droplets of the fluid material within the barrel 50 to pass out of the barrel 50 and around the tip 57 to reach the diaphragm 15 and be expelled through the needle 11. Alternatively, the tip 57 could be have a constant diameter that is formed with one or more grooves extending axially along the tip 57 to permit the passage of liquid medicine from the barrel 50 as the plunger seal 56 bottoms out at the tapered transition portion 54.

The standardized safety needle assembly 10 can be produced in mass production and packaged in a sterile container in a conventional manner. Since the only variation in the safety needle assembly 10 will be the gauge of the needle 11 being selected, the number of needle varieties that must be available is significantly reduced, as each available gauge of needle does not have to be manufactured in each barrel diameter. A barrel size would be selected by the health care practitioner corresponding to the volume of fluid material to be injected into the selected target, then a safety needle assembly 10 having the desired gauge of the needle 11 would be selected, the respective packages opened, and the safety needle assembly 10 connected to the barrel 50 to form the completed syringe.

The application of the safety needle assembly 10 to a particular syringe barrel 50 requires the manual grasping of the housing 12 of the safety needle assembly 10 in one hand and a grasping of the syringe barrel 50 in the other hand, following by a turning motion to engage the housing collar 13 with the connection port 52 to attach the housing 12 of the safety needle assembly 10 on the syringe barrel 50. This connection of the safety needle assembly 10 requires only that the collar 13 on the housing 12 be threaded onto the connector port 52 of the barrel 50 by engaging the respective threads and turning. While the concept of a standardized needle assembly diameter that would fit on a connector port having a diameter corresponding to the attachable needle housing would apply to any form of needle assembly, the concept is particularly advantageous with respect to the utilization of safety needle assemblies 10 because of the manufacturing requirements of providing many different diameters of housings that would otherwise be needed to mate with the differently sized syringe barrels 50. Thus the use of safety needle assemblies is further facilitated while minimizing accidental sticks after prior utilization and discouraging and preventing re-use of the needle. In addition, as is disclosed in aforementioned U.S. Pat. No. 6,626,863, the plunger 55 can also be provided with an internal locking mechanism between the plunger 55 and the walls of the barrel 50 to prevent the withdrawal of the plunger 55 from the barrel 50 once completely depressed into the barrel 50.

The invention of this application has been described above both generically and with regard to specific embodiments. Although the invention has been set forth in what is believed to be the preferred embodiments, a wide variety of alternatives known to those of skill in the art can be selected within the generic disclosure.

It will be understood that changes in the details, materials, steps and arrangements of parts which have been described and illustrated to explain the nature of the invention will occur to and may be made by those skilled in the art upon a reading of this disclosure within the principles and scope of the invention. The foregoing description illustrates the preferred embodiment of the invention; however, concepts, as based upon the description, may be employed in other embodiments without departing from the scope of the invention.

Having thus described the invention, what is claimed is:

1. A safety needle assembly for mounting on a syringe barrel to form a safety syringe for the injection of liquid medicine into a patient in response to a linear movement of a plunger within said syringe barrel, comprising:
an outer housing including a mounting end adapted for connection to said syringe barrel;
an actuator including a body portion having a tubular sheath member extending from a first end thereof, said actuator being mounted within said outer housing for axial sliding movement relative to said outer housing between an exposed position and a covered position;
a diaphragm connected to said actuator and being positioned at said mounting end of said outer housing for slidable movement relative to said outer housing, said diaphragm being engagable with said plunger to effect said sliding movement thereof within said outer housing in response to an end movement of said plunger into said syringe barrel; and
a needle axially supported within said outer housing passing through said sheath member and extending through said diaphragm to be in flow communication with said barrel when said outer housing is mounted thereon, said needle having a point that is exposed beyond said sheath member when said actuator is in said exposed position, said point being positioned internally of said sheath member when said actuator is in said covered position.

2. The safety needle assembly of claim 1 wherein said outer housing is formed with a collar at said mounting end having threads formed therein to be engagable with corresponding threads formed on said barrel.

3. The safety needle assembly of claim 1 wherein said body portion of said actuator having a pair of axially extending legs projecting from a second end of said body portion opposite from said first end, said diaphragm being mounted on said legs.

4. The safety needle assembly of claim 3 wherein each of said legs are formed with a protruding lip at a distal end thereof, said diaphragm being formed with cavities having a shape corresponding to said protruding lips to receive said distal ends of said legs to define a positive connection between said diaphragm and said actuator.

5. The safety needle assembly of claim 3 wherein said housing is formed with a central needle support member having a bore extending axially therethrough for the passage of said needle from said actuator to said diaphragm.

6. The safety needle assembly of claim 5 wherein said central needle support includes a transversely extending bridge member, said central needle support having a lower portion positioned on one side of said bridge member and an upper portion positioned on an opposing side of said bridge member, said bore extending through both said upper and lower portions.

7. The safety needle assembly of claim 6 wherein said diaphragm is mounted on said upper portion of said central needle support to guide the sliding movement thereof over said needle within said outer housing.

8. The safety needle assembly of claim 7 wherein said bridge member spans across the diameter of said outer housing leaving an opening on opposing sides of said bridge member for the passage of said legs of said actuator past said central needle support for engagement with said diaphragm.

9. The safety needle assembly of claim 8 further comprising a locking mechanism to lock said actuator in said covered position after said plunger has moved said diaphragm and said connected actuator within said outer housing into said covered position.

10. The safety needle assembly of claim 9 wherein said syringe barrel includes a connection port for detachably mounting said outer housing thereto.

11. The safety needle assembly of claim 10 wherein said connection port is formed with threads on an outer surface thereof, said outer housing having internal threads corresponding to said threads on said connection port.

12. The safety needle assembly of claim 11 wherein said diaphragm is oriented within said connection port when said outer housing is mounted thereto for engagement with said plunger.

13. A safety syringe comprising:
a syringe barrel including:
a hollow cylindrical body defining a specified volume and having an exterior surface and an interior surface, said cylindrical body including an actuation end formed with a gripping flange and a mounting end opposite said actuation end, said cylindrical body being formed with a first generally uniform diameter extending from said actuation end to said mounting end; and
a connection port on said mounting end formed with a quick attach apparatus, said connection port having a second diameter;
a plunger including a seal member engagable with said interior surface, said plunger being linearly movable within said cylindrical body to push liquid within said cylindrical body toward said mounting end, said seal extending into said connection port when said plunger has reached an end position within said cylindrical body;
a safety needle assembly detachably mounted on said quick attach apparatus on said connection port, said safety needle assembly including:
an outer housing formed for detachable mounting on said connection port;
an actuator movably supported within said outer housing and including a tubular sheath member extending outwardly therefrom, said actuator being movable from an exposed position to a covered position;
a diaphragm mounted on said actuator and being movably positioned within said outer housing for engagement with said actuation tip of said plunger when said plunger reaches said end position to effect movement of said actuator from said exposed position to said covered position; and
a needle fixedly supported within said outer housing and passing through said tubular sheath member, through said actuator and through said diaphragm for flow communication with said cylindrical body of said syringe barrel when said safety needle assembly is mounted on said connection port, said needle having a point that is exposed for injection into a target when said actuator is in said exposed position, said point being located within said tubular sheath member when said actuator is in said covered position.

14. The safety syringe of claim 13 wherein said actuator includes a body portion having a pair of axially extending legs projecting oppositely from said sheath member.

15. The safety syringe of claim 14 wherein said housing is formed with a central needle support member comprising:
a transversely extending bridge member;
a lower portion positioned on one side of said bridge member;
an upper portion positioned on an opposing side of said bridge member; and
a bore extending through said central needle support member for the passage of said needle from said actuator to said diaphragm.

16. The safety syringe of claim 15 wherein said diaphragm is mounted on said upper portion of said central needle support to guide the sliding movement thereof over said needle within said outer housing.

17. The safety syringe of claim 16 wherein said bridge member spans across the diameter of said outer housing leaving an opening on opposing sides of said bridge member for the passage of said legs of said actuator past said central needle support for engagement with said diaphragm.

18. The safety syringe of claim 17 further comprising a locking mechanism to lock said actuator in said covered position after said plunger has moved said diaphragm and said connected actuator within said outer housing into said covered position.

19. In a safety syringe having a syringe barrel defining a predetermined volume for the dispensing of liquids via a linear movement of a plunger within said syringe barrel through a needle having an exposed orientation for injection into a patient and a covered orientation in which the needle is prevented from being injected, the improvement comprising:
a safety needle assembly being detachably mountable on said syringe barrel and including said needle, a safety cover apparatus and a mechanism for actuating said safety cover apparatus cooperable with said needle for movement from said exposed orientation to said covered orientation.

20. The safety syringe of claim 19 wherein said safety needle assembly includes an outer housing adapted for detachable connection to said syringe barrel, said safety cover apparatus comprising:
an actuator movably supported within said outer housing and including a tubular sheath member extending outwardly therefrom, said needle being positioned within said sheath member and being fixed relative to said outer housing, said actuator being movable from an exposed position to a covered position.

21. The safety syringe of claim 20 wherein said safety needle assembly further comprises:
a diaphragm mounted on said actuator and being movably positioned within said outer housing for engagement with said plunger when said plunger reaches an end position to effect movement of said actuator from said exposed position to said covered position.

22. The safety syringe of claim 21 wherein said outer housing is formed with a central needle support member comprising:
a transversely extending bridge member;
a lower portion positioned on one side of said bridge member;
an upper portion positioned on an opposing side of said bridge member; and
a bore extending through said central needle support member for the passage of said needle from said actuator to said diaphragm so that said needle is in flow communication with said syringe barrel when said outer housing is mounted on said syringe barrel.

23. The safety syringe of claim 22 wherein said actuator includes a pair of axially extending legs projecting oppositely from a body portion with respect to said sheath member, said bridge member spanning across said outer housing to define an opening on opposing sides of said bridge member for the passage of said legs of said actuator past said central needle support for engagement with said diaphragm.

24. The safety syringe of claim 23 wherein said diaphragm is mounted on said upper portion of said central needle support to guide the sliding movement thereof over said needle within said outer housing.

25. The safety syringe of claim 24 wherein said safety needle assembly further includes a locking mechanism to lock said actuator in said covered position after said plunger has moved said diaphragm and said connected actuator within said outer housing into said covered position.
